(19) United States
(12) Patent Application Publication
(10) Pub. No.: US 2025/0027009 A1
(43) Pub. Date: Jan. 23, 2025

(54) PURIFICATION METHOD FOR ALPHA-SULFO FATTY ACID SALT

(71) Applicant: BASF SE, Ludwigshafen (DE)

(72) Inventors: Ben Chuan Zhu, Shanghai (CN); Fang Qin Lin, Shanghai (CN); Yan Xiao, Shanghai (CN); Shou Dao Zhang, Shanghai (CN); Claudia Brunn, Düsseldorf-Holthausen (DE); Detlev Stanislowski, Düsseldorf-Holthausen (DE)

(21) Appl. No.: 18/714,323

(22) PCT Filed: Nov. 22, 2022
(86) PCT No.: PCT/EP2022/082797

§ 371 Date: May 29, 2024

(30) Foreign Application Priority Data
Dec. 1, 2021 (WO) PCT/CN2021/134796

(51) Int. Cl. C11C 1/00; A61K 8/46; A61Q 19/00

(52) U.S. Cl. CPC C11C 1/007; A61K 8/466; A61Q 19/00; A61K 2800/10

(57) ABSTRACT

The present invention is related to a method for purifying crude α-sulfo fatty acid salt obtained by the reaction of fatty acid and sulfur oxides, comprising the following steps: a) diluting the crude α-sulfo fatty acid in a solvent and adjusting the ph from 2 to 4; b) heating up the slurry of a-sulfo fatty acid salt obtained in the step a) until being fully solubilized: c) cooling down the solution of α-sulfo fatty acid salt obtained in the step b) to room temperature gradually to obtain the crystals; d) filtering the crystal obtained in the step c) to remove the motherinsing the filter cake at least twice with acid aqueous solution to obtain the purified α-sulfo fatty acid salt.

### TECHNICAL FIELD

The present invention is in relation to a method for purifying crude alpha-sulfo fatty acid salts obtained by the reaction of fatty acid and sulfur oxides. The method of the present invention can effectively and efficiently remove the impurity of sulfate.

### BACKGROUND ART

The sulfonation of organic acids or alcohols for the production of anionic surfactants has been known for a long time. For example, chlorosulfonic acid, sulfur trioxide or concentrated sulfuric acid can be used as sulfonating reagents. It is known that α-sulfo fatty acid salts (i.e., a mixture of salt forms, typically mono-and disalts), in particular disalts based on fatty acids with a chain length of 12 to 18 carbon atoms, are distinguished by good cleaning properties. In the past a-sulfo fatty acids or their salts have been prepared by sulfonation of fatty acids using anhydrous sulfur trioxide or sulfur dioxide as sulphonating agent. The acids have either been recovered as such or converted to the salts by neutralization or by precipitation in the presence of more soluble alkaline salts such as the sulfate. The product obtained by these processes have generally been colored due to the presence of sulfate and a bleaching step, for example with sodium hypochloride or compounds producing hypochlorite ions in solutions is needed to have less colored product, but the product still contained sulfate which is not desirable to the consumer. The personal care products are driven by consumer needs. Free-from claim are continuing to grow and sulfate-free is a leading claim for this trend as there have been growing concerns in the marketplace over the negative effects of the sulfate-containing compositions which are widely regarded as a potential irritant for eyes and skin.

GB1214714 A disclosed a process for preparing monosodium alpha-sulphocarboxylic acids. The process offers the purified α-sulfofatty acid salts by solvent evaporation and filtration with water and air-blowing. Even the product obtained by this process is a white filter cake and the bleaching step is not necessary to be employed, but the product still contains sulfate which cannot be removed by using this process.

US patent application 2020/0100998 A1 discloses a method for producing a light-coloured alpha-sulfo fatty acid disalts which comprises the bleaching of neutralized aqueous pastes of sulfonation products of fatty acids with hydrogen peroxide. However, the method is aimed to obtain alpha-sulfo fatty acid salts with an acceptable color level without any process or treatment for purifying the crude alpha-sulfo fatty acid salts to achieve a lower level of sulfate content.

According to the previous state of art, however, there was no readily accessible method for obtaining purified α-sulfo fatty acid salts with acceptable level of content of sulfate salt. The object of the invention is therefore to develop a method for purifying crude a-sulfo fatty acid salts obtained by the reaction of fatty acid and sulfur oxides.

### SUMMARY OF INVENTION

The surprising discovery has been made by the present invention, that purified α-sulfo fatty acid salts can be obtained with very low content of sulfate by the purification method of the present invention.

In one aspect, the present invention is related to a method for purifying crude α-sulfo fatty acid salts obtained by the reaction of fatty acid and sulfur oxides, comprising the following steps:
a) diluting the crude α-sulfo fatty acid salts in a solvent and adjusting the pH from 2 to 4;
b) heating up the slurry of α-sulfo fatty acid salts obtained in the step a) until being fully solubilized;
c) cooling down the solution of α-sulfo fatty acid salts obtained in the step b) to room temperature gradually to obtain the crystals;
d) filtering the crystals obtained in the step c) to remove the mother liquor; and
e) rinsing the filter cake at least twice with acid aqueous solution to obtain the purified α-sulfo fatty acid salts.

Preferably, in the step a), the pH value is adjusted with hydrochloride acid to from 2 to 4, preferable from 2.5 to 3.5.

Preferably, the weight ratio of the alpha-sulfo fatty acid salts to the solvent in the step a) is in the range of 1:20 to 1:4, preferably in the range of 1:18 to 1:5, preferably in the range of 1:16 to 1:6, more preferably in the range of 1:15 to 1:7, still more preferably in the range of 1:13 to 1:8.

Preferably, the acid aqueous solution used in the step e) at least comprises citric acid in an amount of 0.001% to 5%, preferably in an amount of 0.01% to 3%, still preferably in an amount of 0.03% to 2.5%, more preferably from 0.05% to 2% by weight.

Preferably, the filter cake is rinsed twice in the step (e). Preferably, the weight ratio of the acid aqueous solution used in the two rinses is from 1:1 to 1:3.5, preferably from 1:1.5 to 1:3, more preferably from 1:1.8 to 1:2.5.

In another aspect, the present invention is in relation to purified α-sulfo fatty acid salts obtained or obtainable by the purification method.

In still another aspect, the present invention is in relation to a personal care composition comprising the purified α-sulfo fatty acid salts obtained by the purification method of the present invention.

Surprisingly and unexpectedly, the inventors have now found that a purified a-sulfo fatty acid salts with very low content of sulfate can be obtained by using the present purification method and thus the objective as outlined above is achieved.

### DESCRIPTION

Throughout the description, including the claims, the term “comprising one” or “comprising a” should be understood as being synonymous with the term “comprising at least one”, unless otherwise specified, and “between” should be understood as being inclusive of the limits.

The terms “a”, “an” and “the” are used to refer to one or to more than one (i.e., to at least one) of the grammatical object of the article.

The term “and/or” includes the meanings “and”, “or” and also all the other possible combinations of the elements connected to this term.

The term “wt. %” or “weight percent” used herein, refers to the ratio of the weight of a particular component to the total weight of the whole item multiplied by 100.

The term “substantially” as used herein refers to at least 80%, 90%, 95%, 99% or 100%.

It should be noted that in specifying any range of concentration, weight ratio or amount, any particular upper concentration, weight ratio or amount can be associated with any particular lower concentration, weight ratio or amount, respectively.

The present invention is related to a method for purifying crude α-sulfo fatty acid salts obtained by the reaction of fatty acid and sulfur oxides, comprising the following steps:
a) diluting the crude α-sulfo fatty acid salts in a solvent and adjusting the pH from 2 to 4;
b) heating up the slurry of α-sulfo fatty acid salts obtained in the step a) until being fully solubilized;
c) cooling down the solution of α-sulfo fatty acid salts obtained in the step b) to room temperature gradually to obtain the crystals;
d) filtering the crystals obtained in the step c) to remove the mother liquor; and
e) rinsing the filter cake at least twice with acid aqueous solution to obtain the purified α-sulfo fatty acid salts.

According to the state of the art, the process for making an alpha-sulfo carboxylic acid by the reaction of a substantially saturated or unsaturated fatty acid having 8 to 22 carbon atoms with sulfur dioxide or sulfur trioxide comprising (a) mixing solutions (in a substantially inert solvent) of the sulfur oxides and of the fatty acid in amounts such that the sulfur oxides is in molar excess (as would be expected by one skilled in the art, 1.1 to 1.5 moles of sulfur oxides per mole of fatty acid are normally appropriate); (b) maintaining the mixture at a reaction temperature up to the boiling point of the mixture for a time sufficient for substantially complete reaction; (c) thereafter partially neutralizing the reaction mixture by mixing it with an aqueous solution of alkali hydroxides, preferably sodium hydroxide; (d) removing the solvent from the neutralized solution to obtain the crude alpha-sulfo fatty acid salts.

In principle, the sulfonation can be carried out by all known processes. However, it is preferred to carry out the sulfonation in a Fallfil reactor and with sulfur dioxide or sulfur trioxide as the sulfonating reagent. Such a method is described in detail, for example in German patent application 40 35 935.

The fatty acid suitable for use in the production process are substantially linear or branched, saturated or unsaturated fatty acids having 6 to 22 carbon atoms, and especially 8 to 18 carbon atoms. A fatty acid of which at least 90% by weight is lauric, stearic, myristic or palmitic acid, or a mixture thereof is particular suitable. There are preferably technical fatty acid mixtures such as coconut, palm kernel or tallow fatty acid mixtures, and unsaturated fatty acids, for example oleic acid, may be present in small amounts.

The sulfur oxides can be either sulfur dioxide or sulfur trioxide, which is most conveniently in the form of a stabilized liquid, although it may be employed in gaseous form. The solvent may be any which is substantially inert to the sulfur oxides, the fatty acid and the products of the reaction, and which is a solvent for both sulfur oxides and fatty acid. Preferred solvent includes, for example, dicholormethane as described in the patent GB 1214714 A. The sulfonation of fatty acid can be carried out in a reaction vessel with stirrer and with heating for example to 65° C. at atmospheric pressure or higher pressure. The reaction time to achieve substantially complete reaction may be quite short, for example from 5 minutes to 30 minutes. In some embodiments, longer reaction time may be required if the reaction temperature is lower.

According to the state of the art, the process for making an alpha-sulfo fatty carboxylic acid salt can be performed with gaseous sulfur oxides in the absence of solvent. For example, fatty acids firstly are reacted with gaseous sulfur trioxide. In this case, the sulfur trioxide is preferably used in an amount, in that the molar ratio of SO₃ to fatty acids is in the range from 1.0:1.0 to 1.5:1.0. The fatty acids are introduced in this case into the reactor at a reservoir temperature in the range of 70 to 95° C. Preferably, the liquid sulfonation product obtained after the sulfonation is maintained and aged at this temperature for 5 to 20 minutes in a temperature-controlled postreaction coil.

The crude sulfonated fatty acid products/sulfonation products obtained in this way, which are acidic sulfonation products are then subject to partially or completely being neutralized. The neutralizing agent is used so that sulfonic acid group and carboxylic acid group will be converted or partially converted to their salt form depends on the amount of neutralizing agent. Ammonia, organic bases such as amines, but in particular alkali metal and alkaline earth metal hydroxides, and alkali metal carbonate and alkali metal bicarbonate are suitable as neutralizing agents. The acidic mixture obtained by sulfonation of fatty acid can be neutralized by adding alkaline solution comprising alkali hydroxides, such as sodium hydroxide. In particular, it is preferred to use the neutralizing agents in the form of aqueous solutions. Concentrated sodium hydroxide solutions and potassium hydroxide solutions are particularly preferred. The amount of alkali hydroxide or the pH of the neutralized solution can be adjusted to obtain monosodium salt of sulfocarboxylic acid or disodium salt of sulfocarboxylic acid. Normally, the vast majority of the salt obtained is monosodium salt of sulfocarboxylic acid when the pH is adjusted below 5, preferably below 4.5, and the disodium salt of sulfocarboxylic acid can be obtained when the pH is adjusted higher than 4.5. It is also possible to use hypochlorites of sodium or potassium, preferably in aqueous solution or suspension, as neutralizing agents. In this way, a desired bleaching effect of the products can be achieved at the same time. In particular, however, it is preferred to add bleaching agents to the mono-salt pastes only after they have been produced. It is preferred to bleach the mono salt with an aqueous hydrogen peroxide solution, perborate monohydrate, perborate tetrahydrate or hypochlorite, preferably with a concentrated aqueous hydrogen peroxide solution.

The crude alpha-sulfo fatty acid salts are obtained after neutralization and filtration for removing the liquid, and the sodium sulfate salts are present as impurity. According to any one embodiment of the present invention, the crude alpha-sulfo fatty acid salts comprise more than 4 wt. %, in particular from 6 wt. % to 14 wt. % or from 8 wt. % to 12wt. % of the sulfate (e.g. sodium sulfate) as impurity.

To achieve the objective of the present invention, a method for purifying the crude a-sulfo fatty acid salts obtained by the reaction of fatty acid and sulfur oxides is discovered by the inventors, which comprises the following steps:
a) diluting the crude α-sulfo fatty acid salts in a solvent and adjusting the pH from 2 to 4;
b) heating up the slurry of α-sulfo fatty acid salts obtained in the step a) until being fully solubilized;
c) cooling down the solution of α-sulfo fatty acid salts obtained in the step b) to room temperature gradually to obtain the crystals;
d) filtering the crystals obtained in the step c) to remove the mother liquor; and
e) rinsing the filter cake at least twice with acid aqueous solution to obtain the purified α-sulfo fatty acid salts.

### Step (a)

The crude alpha-sulfo fatty acid salts are diluted in a solvent and the pH is adjusted to from 2 to 4, preferably 2.5 to 3.5, more preferably around 3, in particular, the pH is adjusted preferably with hydrochloride acid (e.g., with the concentration within 28 to 36% by weight as supplied commercially). The suitable solvent to dilute the crude alpha-sulfo fatty acid salts can be water or water-miscible solvent such as acetic acid, acetaldehyde, acetone, acetonitrile, butanediol, ethanol, ethylamine, ethylene glycol, formic acid, glycerol, methanol, propanol, propylene glycol, pentanediol, ethylamine etc. In some embodiment, the solvent can be acidic solution comprising one or more acids including sulfuric acid, hydrochloric acid, acetic acid, citric acid, methanesulfonic acid, p-Toluenesulfonic acid or sulfonic acid. preferably the solvent comprises water, and more preferably the solvent is substantially water.

In some embodiments, the weight ratio of the a-sulfo fatty acid and the solvent is from 1:20 to 1:4, preferably from 1:18 to 1:5, preferably from 1:16 to 1:6, more preferably from 1:15 to 1:7, still more preferably from 1:13 to 1:8.

### Step (b) and Step (c)

A slurry of alpha-sulfo fatty acid salts is obtained in the step (a) and is then heated up until the alpha-sulfo fatty acid salts fully solubilized (step (b)), and then cooled down gradually to room temperature (step (c)) to obtain the crystals of alpha-sulfo fatty acid salts.

In some embodiments of the present invention, the solution of alpha-sulfo fatty acid salts obtained in the step (b) is cooled down to room temperature at a cooling rate of 5 to 40 minutes per degree Celsius, preferably from 5 to 30 minutes per degree Celsium. In some embodiments of the present invention, in the step (c), the solution of alpha-sulfo fatty acid salts is cooled down to from 40 to 60° C., preferably from 45 to 55° C. and this temperature is maintained for at least 1 h, preferably from 1 to 2 or to 2.5 hours. The solution of alpha-sulfo fatty acid salts is then continued being cooled down to room temperature to obtain the crystals.

### Step (d)

The slurry containing crystals of the alpha-sulfo fatty acid salts obtained in the step c) are transferred into a filter press to remove the mother liquor under pressure, for example 2-4 bar, to collect the crystals of the alpha-sulfo fatty acid salts.

### Step (e)

the filter cake comprising the crystals of the alpha-sulfo fatty acid salts obtained in the step (d) was rinsed with filter press operating under pressure (for example 2-4 bar) at least twice with acid aqueous solution to obtain the purified α-sulfo fatty acid salts.

In some embodiments of the present invention, the acid aqueous solution may comprise citric acid, succinic acid, oxalic acid, glycolic acid, tartaric acid, or phosphoric acid, preferably, the acid aqueous solution used in the step e) at least comprises citric acid, more preferably the acid aqueous solution comprises citric acid in an amount of 0.001% to 5%, preferably in an amount of 0.01% to 3%, still preferably in an amount of 0.03% to 2.5%, more preferably from 0.05% to 2% by weight. In some embodiments of the present invention, the weight ratio of the acid aqueous solution used in the step e) to the alpha-sulfo fatty acid salts can be from 40:1 to 1:1, for example from 30:1 to 2:1, from 20:1 to 2.5:1, preferably from 15:1 to 3:1, more preferably from 11:1 to 4:1, still more preferably from 8:1 to 4.5:1, still more preferably from 7.8:1 to 5:1, most preferably from 7.8:1 to 5.6:1.

In some preferred embodiments, the filter cake is rinsed twice with acid aqueous solution and the weight ratio of the acid aqueous solution used in the two rinses is from 1:1 to 1:3.5, preferably from 1:1.5 to 1:3, more preferably from 1:1.8 to 1:2.5, preferably the acid aqueous solution comprises citric acid. In some preferred embodiments, the acid aqueous solution used in the first rinse is a solution of citric acid having pH from 2 to 4, preferably from 2.5 to 3.5, more preferably around 3 and the acid aqueous solution used in the second rinse is citric acid solution with a concentration of from 0.5 wt. % to 4wt. %, preferably 1 wt. % to 3.5 wt. %, more preferably from 1.2 wt. % to 3 wt. %, most preferably from 1.5 wt. % to 2.5 wt. %. The purified a-sulfo fatty acid salts are obtained after the filtration to remove the rinsing solution.

The purified a-sulfo fatty acid salts obtained by the method of the present invention contains less than 2wt. % of sulfate impurity, preferably less than 2 wt. %, more preferably less than 1 wt. % of sulfate impurity.

The present invention also relates to the use of the alpha-sulfo fatty acid salts obtained or obtainable by the process according to the present purification method for personal care or cosmetic compositions and also detergents and cleaners.

The present invention also relates to a personal care composition or a cosmetic composition comprising the purified a-sulfo fatty acid salts obtained by the purification method of the present invention. The personal care compositions intended for topical application to skin or hair. Personal care compositions can be rinse-off formulations, in which the product can be applied topically to the skin or hair and then subsequently rinsed within seconds to minutes from the skin or hair with water. The product can be also be wiped off using a substrate. The personal care composition can be, for example, in the form of a liquid, semi-liquid cream, lotion, gel, solid, or a combination thereof. Preferably, the composition is in the form of hair shampoo, shower gels, soaps, syndets, washing pastes, washing lotions, scrub preparations, foam baths, oil baths, shower baths, shaving foams, shaving lotions, shaving creams, toothpastes, mouthwashes and dental care products.

Examples of personal care compositions can include but are not limited to bar soaps, shampoos, conditioning shampoos, body washes, moisturizing body washes, shower gels, skin cleansers, cleansing milks, in-shower body moisturizers, pet shampoos, shaving preparations, and cleansing compositions used in conjunction with a disposable cleansing cloth. With regard to detergent composition and cleaning composition, the compositions may be, for example, bath and toilet cleaners and the like, and also for cleaning and/or fragrance gels for use in sanitary installations.

The personal care compositions of the present invention may further comprise surfactants, like anionic, nonionic, cationic, amphoteric surfactant, skin actives, skin conditioning agents, and thickening agents.

The personal care compositions of the present invention are generally prepared by conventional methods such as are known in the art of making rinse-off personal care compositions. Such methods typically involve mixing of the ingredients in one or more steps to a relatively uniform state, with or without heating, cooling, application of vacuum, and the like.

### EXAMPLES

### Materials

Crude alpha-sulfo fatty acid salt: Sulfopon® SFA (45wt. % active matter), available from BASF

Citric acid monohydrate: Sinopharm AR, >99.5%

### Measurements

### Composition of Alpha-Sulfo Fatty Acid Salts

10 mg of test sample was dissolved in 10 mL methanol/water (50:50 v/v) and analyzed by liquid chromatography/mass spectrometry coupling. The concentrations of components were determined by the calculation of area % distribution on the chromatography.

### Sulfate Content

100 mg of test samples were weighted in independently in 100 mL volumetric flasks and topped up to the mark with deionized water. The test solutions were analyzed by ion chromatography. The quantification is performed via the peak areas using external standard method.

### Color (Hazen)

The alpha-sulfo fatty acid salts were diluted with a mixture of deionized water (95% by weight) and isopropanol (5% by weight) to a dry residue of 4% by weight-based on the total content of the diluted solution. The color value of this solution was determined in an 11 mm round cuvette in a commercial color measuring instrument from Hach Lange GmbH (Lico 500). The color value is stated in Hazen units.

The purification method is detailed as below:

Crude alpha-sulfo fatty acid salts (Sulfopon® SFA) were diluted in water to obtain a slurry of 700 g (alpha-sulfo fatty acid salt concentration from 8% to 15% by weight) and the pH value was adjusted to pH 3 with hydrochloride acid or citric acid. The slurry was then heated up to fully solubilize the alpha-sulfo fatty acid salt and then cooled down to room temperature with the below program:
Heating up from room temperature to 70° C. in 3.5 hours and maintaining at 70° C. until fully solubilization of the crude alpha-sulfo fatty acid salt;
cooling down to 60° C. at the rate of 6 minutes per degree Celsium and maintaining at 60° C. for 1 hour;
then cooling down to 50° C. at the rate of 30 minutes per degree Celsium and maintaining at 50° C. for 1 hour;
then cooling down to 40° C. at the rate of 30 minutes per degree Celsium and maintaining at 40° C. for 1 hour;
then cooling down to room temperature at the rate of 6 minutes per degree Celsium.

The slurry containing crystals of alpha-sulfo fatty acid salts was transferred into a filter press (SHXB-BZ-0.5L) to remove mother liquor under 2 bar for 10 min. The filter cake was rinsed and filtered as below detailed in Table 1. The samples were prepared by using different pH adjusters and the rinsing programs.

TABLE 1

| | Preparation of slurry of the | | | | |
|---|---|---|---|---|---|
| | alpha-sulfo fatty acid salts | | | | |
| | Concentration | | | | |
| | of the α-sulfo | | | | |
| | fatty acid salt | | Rinsing program | Filtration and | Color |
| | (active matter) | pH adjuster | and filtration | rinsing time | (Hazen) |
| 1 | 15 wt. % | Citric acid | Only one rinsing step | Fully blocked | >1000 |
| | | | with 200 g of citric | | |
| | | | acid solution at pH of | | |
| | | | 3 | | |
| 2 | 10 wt. % | Citric acid | Only one rinsing step | Blocked after 1 | >1000 |
| | | | with 200 g of citric | hour | |
| | | | acid solution at pH of | | |
| | | | 3 | | |
| 3 | 8 wt. % | Citric acid | Only one rinsing step | 30 hours | 187 |
| | | | with 200 g of citric | | |
| | | | acid solution at pH of | | |
| | | | 3 | | |
| 4 | 8 wt. % | Citric acid | Only one rinsing step | 2 hours | 500 |
| | | | with 200 g of 1.5 wt. % | | |
| | | | NaCl solution | | |
| 5 | 8 wt. % | Citric acid | Only one rinsing step | 4 hours | 500 |
| | | | with 200 g of 0.5 wt. % | | |
| | | | NaCO₃ solution | | |
| 6 | 8 wt. % | Citric acid | First rinsing step with | 1 hour for the | 300 |
| | | | 66 g citric acid solution | first rinsing | |
| | | | at pH of 3 | step | |
| | | | Second rinsing step | 0.1 hour for the | |
| | | | with 134 g of 1.5 wt. % | second rinsing | |
| | | | NaCl solution | step | |
| 7 | 8 wt. % | Citric acid | First rinsing step with | 1 hour for the | 176 |
| | | | 66 g citric acid solution | first rinsing | |
| | | | at pH of 3 | step | |
| | | | Second rinsing step | 2 hours for the | |
| | | | with 134 g of 1.5 wt. % | second rinsing | |
| | | | citric acid solution | step | |
| 8 | 8 wt. % | Hydrochloride | First rinsing step with | 1 hour for the | 83 |
| | | acid | 66 g citric acid solution | first rinsing | |
| | | | at pH of 3 | step | |
| | | | Second rinsing step | 2 hours for the | |
| | | | with 134 g of 1.5 wt. % | second rinsing | |
| | | | citric acid solution | step | |

The results show that when hydrochloride acid was used as pH adjuster to prepare the slurry of the crude alpha-sulfo fatty acid salts and the citric acid solution was used for rinsing the filter cake twice (i.e., the sample 9), the alpha-sulfo fatty acid salts were obtained with lower Hazen color number which indicates higher purity with less impurity.

Composition comparison of the purified alpha-sulfo fatty acid salts (Sample 8) and the crude alpha-sulfo fatty acid salts is showed in Table 2.

TABLE 2

| | Crude alpha-sulfo | | Purified alpha-sulfo | |
|---|---|---|---|---|
| | fatty acid salt | | fatty acid salt | |
| | (Sulfopon ® SFA) | | (Sample 9) | |
| Alpha-sulfo fatty acid salts | 45 | wt. % | 55 | wt. % |
| Unsulfonated fatty acid | 6 | wt. % | <1 | wt. % |
| Sodium sulfate | 5.6 | wt. % | <1 | wt. % |
| Water | To 100 | wt. % | To 100 | wt. % |

| | | |
|---|---|---|
| Hazen color | >1000 | 83 |

1. A method for purifying a crude a sulfo fatty acid salt obtained by a reaction of a fatty acid and sulfur oxides, comprising:
a) diluting the crude α-sulfo fatty acid in a solvent and adjusting a pH to from 2 to 4;
b) heating a slurry of α-sulfo fatty acid salts obtained in step a) until fully solubilized;
c) cooling a solution of α-sulfo fatty acid salts obtained in step b) to room temperature gradually to obtain crystals;
d) filtering the crystals obtained in step c) to remove a mother liquor; and
e) rinsing a filter cake at least twice with an acid aqueous solution to obtain the purified α-sulfo fatty acid salts.

2. The method according to claim 1, wherein a weight ratio of the α-sulfo fatty acid salts to the solvent in step a) is from 1:20 to 1:4.

3. The method according to claim 1, wherein in step a), the solvent is water or a water-miscible solvent.

4. The method according to claim 1, wherein in step a), the pH is adjusted with hydrochloric acid to 2.5 to 3.5.

5. The method according to claim 1, wherein in step c), the solution of a-sulfo fatty acid salt is cooled to from 40 to 60° C., and maintained for at least 1 h in step c).

6. The method according to claim 1, wherein the acid aqueous solution used in step e) at least comprises citric acid in an amount of 0.001% to 5%.

7. The method according to claim 1, wherein in step e), a weight ratio of the acid aqueous solution to the alpha-sulfo fatty acid salts is from 40:1 to 1:1.

8. The method according to claim 1, wherein the filter cake is rinsed twice with the acid aqueous solution in step e).

9. The method according to claim 8, wherein a weight ratio of the acid aqueous solutions in the at least two rinses is from 1:1 to 1:3.5.

10. The method according to claim 1, wherein the crude a-sulfo fatty acid salts comprise sulfate as impurity.

11. A purified α-sulfo fatty acid salt obtained by the method according to claim 1.

12. A personal care composition comprising the purified α-sulfo fatty acid salt obtained by the method according to claim 1.

13. The personal care composition according to claim 12, wherein the composition is in the form of hair shampoo, shower gels, soaps, syndets, washing pastes, washing lotions, scrub preparations, foam baths, oil baths, shower baths, shaving foams, shaving lotions, shaving creams, and dental care products.
